(19) United States
(12) Patent Application Publication
(10) Pub. No.: US 2007/0249044 A1
(43) Pub. Date: Oct. 25, 2007

(54) Microstructures in three dimensional gel suspensions for growth of cells

(75) Inventors: Tejal A. Desai, San Francisco, CA (US); Brenda Russell, Evanston, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: UNIVERSITY OF ILLINOIS AT CHICAGO, Chicago, IL (US)

(21) Appl. No.: 11/699,840

(22) Filed: Jan. 30, 2007

Related U.S. Application Data
(60) Provisional application No. 60/763,863, filed on Jan. 30, 2006.

(51) Int. Cl. C12N 5/06

(52) U.S. Cl. 435/325

(57) ABSTRACT

The present invention provides a three-dimensional cell culture system for the growth of cells and the method of making the same.

The present application claims benefit of U.S. Provisional Application No. 60/763,863 filed Jan. 30, 2006. This invention was made with government support under HL 64956 awarded by the National Institutes of Health. The government has certain rights in the invention.

### FIELD OF THE INVENTION

The present invention relates to a three-dimensional cell culture system for the growth of cells and the method of making the same. More specifically, the cell culture system comprises three dimensional structures suspended in cell culture gels to provide a three-dimensional environment for cell growth.

### BACKGROUND OF THE INVENTION

There is an enormous demand for cell-based products as well as cell-based therapies. This demand presents an ever-increasing need for a more efficient, robust and “real-world” methods of growing mammalian cells. Generally, it is recognized that eukaryotic cells are slow growing and vulnerable to injuries caused by shear stress and contamination. The vast majority of eukaryotic cells are anchorage-dependent and require a growth surface to attach to in order to proliferate and grow.

One of the major challenges in engineering tissues is mimicking the complex cellular organizations and functions of the native tissues of the human body. The use of a three-dimensional extracellular matrix has been shown to provide an environment for cells that brings about more in vivo like morphology, organization and gene expression. 3D matrices have also provided new insight into tissue organization and more accurate models for pathogenesis than traditional 2D culture techniques, such as Petri dishes. Work done studying human breast cancer has shown the importance of moving from traditional, nonphysiological 2D systems to 3D systems. Culture of human breast epithelial cells in collagen gels has demonstrated the ability of the cells to re-express their in vivo organization and differentiation, and recapitulate histology. These studies also allowed for the identification of previously unknown phenomena at many different levels; cell-cell interactions, gene expression and ECM affects on cellular organization and polarity, which were not present in traditional 2D cultures. 3D gel culture systems also facilitate natural, complex 3D tissue development in vivo. The importance of culturing cells in 3D artificial matrices to mimic the body's own extracellular matrix (ECM) is becoming more apparent, and this is the basis of the current application. ECM is the natural scaffold material in vivo that maintains the 3D tissue architecture, controls cell proliferation, and regulates the processes of cell motility and cell migration (Gumbiner, Cell Feb. 9, 1996;84(3):345-57 1996; Raines, Int J Exp Pathol. June 2000;81(3):173-82).

The third dimension has numerous advantages for cells. Cells show a more lifelike gene expression profile and subcellular architecture when grown in 3D instead of conventional flat 2D surfaces (Cukierman et al., Science. Nov. 23, 2001;294(5547):1708-12; Pedrotty et al., Am J Physiol Heart Circ Physiol. April 2005;288(4):H1620-6). Cocultures of human umbilical-vein epithelial cells and 10T1 I2 mesenchyrnal cells in collagen-Cells in the body respond dynamically to their microenvironment. The composition and physical properties of the extracellular matrix (ECM) are known to modulate both cellular fate and function decisions. The controlled interaction of cells with specific ECM architectures is critical to maintaining cell phenotype. Thus, a much sought after goal in tissue engineering is the fabrication of three-dimensional (3D) tissue scaffolds that incorporate microscale chemical and mechanical domains. Such an approach requires control over matrix architecture in a hierarchical multiscale manner.

The fabrication of controlled complex tissue scaffolds has proven to be more difficult to produce compared to homogeneous porous polymer scaffolds conventionally employed in tissue engineering. Typically, bulk properties of a matrix are adjusted so that the entire cell population can be modulated. However, such bulk changes may not be appropriate when trying to recreate the complex three-dimensional (3D) patterning, organization and regional architecture of one or more cell types in an engineered tissue construct. In such cases, techniques for controlling the local microenvironment presented to specific cell populations within a 3D construct may be desired.

It is known that cells respond to changes in mechanical cues such as gel elasticity, porosity and density [Pelham et al., Biol Bull 1998: 194(3):348-350; Helary et al., Biomaterials May 2005;26(13):1533-1543]. Matching the elasticity of the matrix to a target tissue has shown to cause tissue specific differentiation of neurons [Georges et al., Biophys J Apr. 15, 2006;90(8):3012-3018], myoblasts [Engler et al., J Cell Biol Sep. 13, 2004;166(6):877-887] and stem cells [Engler et al., Cell Aug. 25, 2006;126(4):677-689]. Biochemical cues such as the presence and density of attachment enhancing peptides (e.g. RGD) can also be used to control cells and alter growth parameters [Kurihara et al., J Biosci Bioeng July 2005;100(1):82-87]. A technique is needed that can provide discrete mechanical or biological cues to individual cells in a 3D construct in order to control their growth and differentiation.

Two-dimensional (2D) fabrication techniques (e.g. photolithography, soft lithography) are widely used methods for controlling the microenvironment of cells on planar surfaces. These techniques are used to create cell-scale features that physically guide the cells [Li et al., Crit Rev Biomed Eng 2003;31(5-6):423-488; Whitesides et al., Annu Rev Biomed Eng 2001;3:335-373; Norman et al., Ann Biomed Eng January 2006;34(1):89-101; Folch et al., Annu Rev Biomed Eng 2000;2:227-256]. With proper design of the microenvironment, cells can be coaxed into growing and behaving in a more physiologic manner. This has been previously demonstrated with a wide variety of cell types including: primary cardiac fibroblasts and cardiomyocytes [Boateng et al., Am J Physiol Cell Physiol July 2003;285(1):C171-182; Deutsch J, et al., J Biomed Mater Res 2000;53(3):267-275; Motlagh et al., J Biomed Mater Res A Oct. 1, 2003;67(1):148-157; Motlagh et al., Biomaterials June 2003;24(14):2463-2476], primary hepatocyte cultures [Khetani et al., Hepatology September 2004;40(3):545-554; Park et al., Biotechnol Bioeng Jun. 5, 2005;90(5):632-644], osteoblasts [Popat K C et al., Biomaterials August 2005;26(22):4516-4522; Swan et al., J Biomed Mater Res A Mar. 1, 2005;72(3):288-295; Mata et al., Biomed Microdevices December 2005;7(4):281-293] and smooth-muscle cells [Sarkar et al., Acta Biomaterialia 2005;1(1):93; Goessl et al., J Biomed Mater Res October 2001;57(1):15-24; Thakar et al., Biochem Biophys Res Commun Aug. 8, 2003;307(4):883-890.]. The physical patterning of the surface provides spatial control over the cell populations while simultaneously enhancing the biofunctionality.

Various fabrication techniques have recently been described for creating internal structures in 3D gels for cell guidance, including; soft lithography [Norman et al., Tissue Eng March-April 2005;11(3-4):378-386], laser guided direct writing [Nahmias et al., Biotechnol Bioeng Oct. 20, 2005;92(2):129-136], single-photon lithography [Hahn et al., Advanced Materials 2006;18(20):2679-2684] and two-photon laser scanning lithography [Hahn et al., Advanced Materials 2006;18(20):2679-2684; Miller et al., Biotechnol Bioeng Apr. 20, 2006;93(6):1060-1068]. However, these techniques have yielded little insight into how cells respond to discrete microscale features in a 3D environment beyond controlling cell migration or organization. Also, these previous approaches do not lend themselves to in vivo administration.

There is a need to control the microscale environment in 3D matrices for tissue engineering applications. This is a challenging but necessary goal and the techniques available thus far have not met this challenge as the techniques used are typically not applicable to in vivo environments

### SUMMARY OF THE INVENTION

In controlling tissue growth in vitro as well as in implants, there is a need to control the forces that an individual cell senses in a 3D matrix in order to allow for local control over microenvironmental cues in an extracellular matrix. To address such needs, there is described herein a three-dimensional cell culture matrix comprising composite of a cell culture gel and polymeric microstructures suspended in the cell culture gel, wherein the microstructures have a cross sectional area of between 1 μm² to 2500 μm² and are randomly suspended in the cell in a concentration of from 10 to 100,000 microstructures/ml of gel, wherein the microstructures form a scaffold for the cells. The cross-sectional area may be, for example 1 μm², 2 μm², 5 μm², 10 μm², 15 μm², 20 μm², 25 μm², 30 μm², 35 μm², 40 μm², 45 μm², 50 μm², 55 μm², 60 μm², 65 μm², 70 μm², 75 μm², 80 m², 85 μm², 90 μm², 95 μm², 100 μm², 110 μm², 120 μm², 130 μm², 140 μm², 150 m², 160 μm², 170 μm², 180 μm², 190 μm², 200 μm², 250 m², 300 μm², 350 μm², 400 μm², 450 μm², 1 μm², 500 μm², 600 μm², 700 μm², 800 μm², 900 μm², 1000 μm². Of course, it should be understood that the cross sectional area my be of a value of any integer between these figures and between the figures 1 μm² to 2500 μm². Likewise, the microstructures per/ml can be 10, 20, 30, 40, 50, 60, 70, 80, 90, 100, 150, 200, 250, 300, 350, 400, 450, 500, 550, 600, 650, 700, 750, 800, 850, 900, 950, 1000, etc. and be of a value of any integer between 10 and 100,000. In some preferred embodiments, the composite comprises 50,000 microstructures per ml of gel.

In the three-dimensional cell culture matrix the cell culture gel can be any gel typically employed for cell culture and is preferably selected from the group consisting of Matrigel, an alginate gel, a collagen gel, and an agarose gel, or a synthetic hydrogel. In some embodiments, the gel may further comprise one or more of laminin, fibrin, fibronectin, proteoglycans, glycoproteins, glycosaminoglycans, chemotactic agents, or growth factors.

The microstructures in the composite may be made of non-biodegradeable or biodegradeable polymers. In certain embodiments, the microstructures are comprised of collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbornates, poly(ethylene oxide), co-polymers of the above, mixtures of the above, and adducts of the above. In particular embodiments, the microstructures comprise a mixture of poly(L-lactic acid) and poly(lactic acid-co-glycolic acid). In some embodiments, the microstructures are made up of co-polymers of two or more polymers.

In some embodiments, substantially all of the microstructures are comprised of a single polymer. In other embodiments, the microstructures form a mixed population of microstructures made of different polymers. The microstructures can be of a uniform size and shape or alternatively may be of different sizes and shapes and randomly dispersed throughout the gel. In some embodiments, the microstructures are made of different polymers materials of different stiffnesses.

In certain embodiments, one or more of the microstructures further comprise a cell-adhesion promoting agent on the surface of the microstructures. Such agents include but are not limited to agents selected from fibronectin, integrins, extracellular matrix or peptides, and oligonucleotides that promote cell adhesion. In additional embodiments, it is the gel that further comprises agents to facilitate cell adhesion and cell growth selected from the group consisting of further comprises one or more of laminin, fibrin, fibronectin, proteoglycans, glycoproteins, glycosaminoglycans, chemotactic agents, or growth factors. Of course, it is contemplated that both the gel and the microstructures may comprise agents that facilitate cell adhesion and/or growth. Exemplary growth factors for use in the composites include but are not limited to activin-A (ACT), retinoic acid (RA), epidermal growth factor, bone morphogenetic protein, TGF-β, hepatocyte growth factor, platelet-derived growth factor, TGF-β, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), mechano-growth factor (MGF), muscle morphogenic factor and stem cell growth factors.

In still other embodiments, the microstructure has encapsulated therein an agent, for example, a drug, therapeutic peptide, nucleic acid, dye and the like, to be delivered to a target site in vivo.

In specific embodiments, the composite contains microrods as the microstructures where the microrods have dimensions of 100 μm×15 μm×15 μm. In specific embodiments, the three-dimensional cell culture matrix is one in which the ratio of microstructures to cells is between one to one and 100 to one. It is contemplated that the ratio of microstructures:cells is 2:1; 3:1; 4:1, 5:1; 6:1; 7:1; 8:1; 9:1; 10:1; 11:1; 12:1; 13:1; 14:1: 15:1; 16:1; 17:1; 18:1; 19:1; 20:1; 21:1; 22:1; 23:1; 24:1; 25:1; 26:1; 27:1; 28:1; 29:1; 30:1; 31:1; 32:1; 33:1; 34:1; 35:1; 36:1; 37:1; 38:1; 39:1; 40:1; 41:1; 42:1; 43:1; 44:1; 45:1; 46:1; 47:1; 48:1; 49:1; 50:1; 51:1; 52:1; 53:1; 54:1; 55:1; 56:1; 57:1; 58:1; 59:1; 60:1; 61:1; 62:1; 63:1; 64:1; 65:1; 66:1; 67:1; 68:1; 69:1; 70:1; 71:1; 72:1; 73:1; 74:1; 75:1; 76:1; 77:1; 78:1; 79:1; 80:1; 81:1; 82:1; 83:1; 84:1; 85:1; 86:1; 87:1; 88:1; 89:1; 90:1; 91:1; 92:1; 93:1; 94:1; 95:1; 96:1; 97:1; 98:1; 99:1; 100:1.

The invention also contemplates a cell culture of system comprising a population of cells to be cultured seeded in the gel of a three-dimensional matrix as described above. The cells may be any cells that are capable of anchorage dependent growth and include but are not limited to cells selected from the group consisting of endothelial cells, myoblasts, cardiomyocytes, stem cells, skeletal muscle cells, smooth muscle cells, fibroblasts, a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell.

Typically, the cells are seeded at an initial concentration of from about 250×10³ cells/μm³ gel to about 1000×10³ cells/μm³ gel.

Also contemplated is a method of growing cells in culture comprising: seeding a population of cells in the gel of a three-dimensional matrix according to the invention and culturing the seeded cells in a cell culture medium that supports the growth of the cells of the population.

### DETAILED DESCRIPTION OF PREFERRED EMBODIMENTS

There is a need for three dimensional present invention provides methods and compositions of a composite cell culture system that is made of microstructures suspended in a cell culture gel matrix is able to modulate the local microenvironment of cells grown in that composite cell culture, leading to alterations in cell proliferation. The presence of these microstructures suspended in the gel provide a discrete, cellular-scale zone of increased mechanical stiffness within the gel. It is suggested that as a cell migrates through the gel its migration will be resisted by the presence of a microstructure in the gel leading to functional modulation, including changes in proliferation and cytoskeletal organization. The present invention exploits the use of free-floating microstructures within a cell culture gel to produce a three-dimensional and microstructural cell guidance microenvironment for controlling cell-cell interactions. This may be used to engineer superstructures (e.g., tissue patches and the like) of cells that more closely resemble those of the human body. In exemplary embodiments, the cell used are heart muscle cells as these cells are well-characterized so that many physiological and biochemical markers are available for determination of success of phenotypic maturation and connectivity. However, it should be understood that the three dimensional cell culture systems of the invention may be employed for the growth of any anchorage dependent cell.

By way of background explanation, it is noted that reactive forces are important to cell growth in a three-dimensional environment. Greater reactive forces experienced by cells when a solid, well-anchored structure is encountered are instrumental in directing their morphology and gene expression profile. We systematically vary the parameters of the physical microenvironment surrounding cells and measuring the biological responses: Specifically, alter the reactive forces by including microrods of varying lengths and stiffnesses to vary resistances within the gel (e.g. a larger object has greater resistance to cell forces than smaller object)

The rationale for inclusion of microstructures was the inventor's finding that fibroblasts failed to proliferate when stopped by rigid microprojections but not when they could move small spheres in their path (Boateng et al., 2003). We suggested that fibroblast proliferation depended not simply on the 10 μM vertical microtexture but also on the action-reaction force pairs that exist between two objects (Newton's Third Law). Since fibroblasts are hindered by the extent of local reactive forces, proliferation of the fibroblasts will decrease proportional to resistance, manipulated by the length of the microstructure. Therefore, it is reasoned that reactive forces experienced within a cell will be greater when a larger microstructure is encountered, and fibroblasts will decrease their proliferation proportionately.

The probability that a cell encounters a microstructure in a given three-dimensional cell culture system of the invention will depend on the concentration and volumes of cells and of the microstructures used in that particular system. For example, fibroblasts are relatively small cells (under 1,000 μm³) while myocytes are at least threefold larger. In exemplary embodiments herein, various seeding concentrations of fibroblasts are used (for example, 250, 500 and 1,000×10³ per μm of Matrigel) and various microstructure concentrations from 0, 5,000, 10,000, 20,000, 50,000 and 100,000 microstructures per microliter of gel. While these are exemplary concentrations, they are in the right ranges for optimal concentrations for frequency of interactions. In specific exemplary embodiments, the microstructures are microrods and are microfabricated from SU-8 (a photo-imagable polymer) that has previously been shown to be non cytotoxic in culture (Tao et al. 2005).

### CELLS GROWN IN THE COMPOSITES OF THE INVENTION

In the exemplary studies described in further detail in the examples herein below, fibroblasts grown in the composite structures of the invention that contain either microrods or microcubes. Typically, fibroblasts grown in a Matrigel matrix take on the appearance of fibroblasts in a thick layer of Matrigel, migrating and spreading in culture. However, inclusion of microstructures dramatically changes the appearance of the fibroblasts to become more polarized. A single fibroblast or a group of fibroblasts, can grow along side a microrod with the long-axis of the cell following the long axis of the rod. Alternatively, some cells have no apparent interaction with microrods. Often, the fibroblasts are seen to attach at one end to a microrod with their other end spanning to another microrod. In this manner a large tissue-like complex of interconnected cells and microstructures is built in which some of the cells are anchored and others are more distal.

The microstructures suspended in the gel are able to inhibit cell division. As a control, cells were grown in Matrigel alone at the suggested concentration (BD Biosciences, Bedford, Mass.). This Matrigel system has a low shear modulus of 34 Pa (Semler et al., 2000) allowing effective study of the effects of the randomly distributed microrods without any appreciable resistance from the gel. The effects of the inclusion of microrods on fibroblast proliferation from samples was assessed over a 5 day period in culture. Cells were released from the Matrigel matrix using BD Biosciences Cell Recovery Solution and then counted using a hemocytometer. Cell division was seen to be rapid for cells suspended in Matrigel alone but 5 days the inclusion of 100 μm SU-8 microrods reduced cell number (p<0.06, n=4).

Similarly, rat neonatal myocytes also have been grown herein with microrods in Matrigel. In the presence of microrods, the myocytes form clusters around them by 3 days that span large regions creating a syncytium. Visible synchronous, isometric beating is apparent throughout these large regions of interconnected myocytes through the numerous microrods suspended in the gel. In the gel alone, myocytes remain spherical forming small and isolated clusters of the cells, some of which were beating non-synchronously. The presence of actin-positive striations was confirmed with confocal microscopy and the presence of myocytes alongside the microrods was detected. In lower concentrations of Matrigel, the myocytes are able to move the microrods like a pendulum with a steady detectable rhythm.

The above outlined studies demonstrate that the cell culture systems of the present invention may be used in the culturing cells to modulate the appearance and characteristics of the cells grown therein. In addition, the compositions of the invention also will be useful for tissue engineering. To demonstrate the use of the cell culture composition for this embodiments, 100 micron long microrods and high cell density culture was used. These conditions are good for myocyte contractile maturity because they permit a firm anchor for shortening, or isometric contraction. The myocytes grown in this manner are very healthy in appearance, contract well and are contoured by their attachments. The studies showed that myocyte at highest density are well striated and have junctions connecting them to their neighbors. When density and cell/microrod ratio is high, many cells span from one microrod to another, the whole 3D slab is interconnected and forms a randomly organized syncytium. Given the morphology, it is expected that gene expression for mechanical and electrical junction proteins will be similar to that seen in vivo. Also, the morphology is expected to show the classic features of mature mechanical attachments with focal adhesion spots for fibroblasts, and gap junctions reflecting intercalated discs for myocyte in culture. Morphology is confirmed by westerns where connexin-43 protein expression is expected to increase suggesting improved myocyte-myocyte interaction in the presence of microrods.

Confocal microscopy in Z-stack mode was used to image cells in the vertical plane from the bottom of the dish and showed that the initial seeding of cells and microrods is uniform throughout a gel to at least 500 microns depth.

The three-dimensional cell culture system of the present invention may be used for the growth and study of almost all cell types since all cells that form three-dimensional tissue architecture can be expected to behave as the fibroblasts and myocytes have performed in the examples discussed above. This is because cell attachment is a fundamental biological feature that these cells share in common with each other. All cells that form tissues are anchored to their neighbors and the anchorage is an important and universal modifier of gene expression and cell behavior. Thus, the physical environment affects most types of cell cultures because most cells are anchorage-dependent. Cell types include all that form the organs in the major body systems, notably cardiovascular, digestive, respiratory, urinary, skeletal, muscular, integumentary, nervous, endocrine and reproductive systems. The exceptions are cells that migrate such as the immune and blood cell, and various metastatic cancers.

In some embodiments, it is contemplated that the cell culture systems of the invention may be particularly useful in the growth of embryonic stem cells. There have been many studies which have shown that there are substantial difficulties with growing ES cells in Petri dishes. Typically, in the Petri dish, ES cells over-proliferate, show poor mechanical coupling and show lack of maturation. Growth in the composites of the invention will yield ES cells that are ideal for looking at differentiation aspects that will be relevant to other stem cells from human once appropriate techniques have been standardized to generate sufficient cells from alternative sources. It is worth noting here that 3D Matrigel alone retards maturation of cardiac myocyte differentiation, keeping them in an early, immature stage with five-fold more dividing cells as compared to embryonic stem cells grown on traditional 2D surfaces.

In specific embodiments, the cells are selected from the group consisting of connective tissue cells, organ cells, muscle cells, nerve cells, and any combination thereof. In more specific embodiments, the cells are selected from the group consisting of tenocytes, fibroblasts, ligament cells, endothelial cells, lung cells, epithelial cells, smooth muscle cells, cardiac muscle cells, skeletal muscle cells, islet cells, nerve cells, hepatocytes, kidney cells, bladder cells, urothelial cells, chondrocytes, and bone-forming cells. In other preferred embodiments, the tissue engineering method comprises growing the cells on the scaffold in a bioreactor.

The cells that may be seeded into the gels of the three dimensional matrices of the present invention may be derived from commercially available cell lines, or alternatively may be primary cells, which can be isolated from a given tissue by disaggregating an appropriate organ or tissue which is to serve as the source of the cells being grown. This may be readily accomplished using techniques known to those skilled in the art. Such techniques include disaggregation through the use of mechanical forces either alone or in combination with digestive enzymes and/or chelating agents that weaken cell-cell connections between neighboring cells to make it possible to disperse the tissue into a suspension of individual cells without appreciable cell breakage. Enzymatic dissociation can be accomplished by mincing the tissue and treating the minced tissue with any of a number of digestive enzymes either alone or in combination. Digestive enzymes include but are not limited to trypsin, chymotrypsin, collagenase, elastase, and/or hyaluronidase, Dnase, pronase, etc. Mechanical disruption can also be accomplished by a number of methods including, but not limited to the use of grinders, blenders, sieves, homogenizers, pressure cells, or sonicators to name but a few. For a review of tissue disaggregation techniques, see Freshney, Culture of Animal Cells. A Manual of Basic Technique, 2d Ed., A. R. Liss, Inc., New York, 1987, Ch. 9, pp. 107-126.

Once the primary cells are disaggregated, the cells are separated into individual cell types using techniques known to those of skill in the art. For a review of clonal selection and cell separation techniques, see Freshney, Culture of Animal Cells. A Manual of Basic Techniques, 2d Ed., A. R. Liss, Inc., New York, 1987, Ch. 11 and 12, pp. 137-168. Media and buffer conditions for growth of the cells will depend on the type of cell and such conditions are known to those of skill in the art.

In certain embodiments, it is contemplated that the cells attached to the composite cell culture of the invention are grown in bioreactors. A bioreactor may be of any class, size or have any one or number of desired features, depending on the product to be achieved. Different types of bioreactors include tank bioreactors, immobilized cell bioreactors, hollow fiber and membrane bioreactors as well as digesters. There are three classes of immobilized bioreactors, which allow cells to be grown: membrane bioreactors, filter or mesh bioreactors, and carrier particle systems. Membrane bioreactors grow the cells on or behind a permeable membrane, allowing the nutrients to leave the cell, while preventing the cells from escaping. Filter or mesh bioreactors grow the cells on an open mesh of an inert material, allowing the culture medium to flow past, while preventing the cells from escaping. Carrier particle systems grow the cells on something very small, such as small nylon or gelatin beads. The bioreactor can be a fluidized bed or a solid bed. Other types of bioreactors include pond reactors and tower fermentors. Any of these bioreactors may be used in the present application for regenerating/engineering tissues in the three-dimensional cell culture composites of the present invention.

Certain tissues that are regenerated herein may be encapsulated so as to allow the release of desired biological materials produced by the cells at the site of implantation, while sequestering the implanted cells from the surrounding site. Cell encapsulation can be applied to all cell types secreting a bioactive substance either naturally or through genetic engineering means. In practice, the main work has been performed with insulin secreting tissue.

As indicated above, the cells that are seeded in the culture systems of the invention may be cell lines or primary cells. In certain preferred embodiments, the cells are genetically engineered or “recombinant” cells that have been modified to express a biologically active or therapeutically effective protein product. Techniques for modifying cells to produce the recombinant expression of such protein products are well known to those of skill in the art. In particular preferred embodiments, the compositions of the invention may be used to form a tissue graft or tissue patch. Such a tissue graft may be an autograft, allograft, biograft, biogenic graft or xenograft. Tissue grafts may be derived from various tissue types. Representative examples of tissues that may be used to prepare biografts include, but are not limited to, rectus sheaths, peritoneum, bladder, pericardium, veins, arteries, diaphragm and pleura. For such grafts the cells may be endothelial cells, ligament tissue, muscle cells, bone cells, cartilage cells. Such cells may be grafted into the compositions of the invention alone or in combination with a drug or biologically active agent to be delivered to an in vivo site. For example, such cells for the biograft may be harvested from a host, loaded with the agent of interest and then applied in a perivascular manner at the site where lesions and intimal hyperplasia can develop. Once implanted, the agent of interest (e.g., paclitaxel) is released from the graft and can penetrate the vessel wall to prevent the formation of intimal hyperplasia at the treatment site. In certain embodiments, the biograft may be used as a backing layer to enclose a composition (e.g., a gel or paste loaded with anti-scarring agent).

The cells seeded on the composites of the invention may be any anchorage dependent cells. For example, the cells may be vasculature endothelial cells, parenchymal cells, cells forming cartilage or bone, muscle cells, and nerve cells. In specific embodiments, the parenchymal cells are derived from organs selected from the group consisting of heart, liver, pancreas, intestine, kidney, reproductive tissues and lung. In certain aspects the cells may be pluripotent cells such as for example, those derived from one or more of the group consisting of embryonic stem (ES) cells, early primitive ectoderm-like (EPL) cells in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer.

### MICROSTRUCTURES FOR USE IN THE COMPOSITES OF THE INVENTION

In the present invention microstructures are employed that have approximately the same square cross-sectional area (15×15 μm²) but varying in length (15 μm, 50 μm and 100 μm) and prepared as described herein. The microstructures used in the invention may be of various dimensions ranging from 1-100 microns. The shape of the microstructure can be varied and encompasses rod-like, cuboidal, columnar, spherical, and elliptical. Within a given matrix materials or gel, the microstructures can be suspended in concentrations ranging from 0 to 100,000 microstructures/microliter of gel. They can be suspended evenly or homogeneously throughout the gel or alternatively they can be suspended at varying degrees of density in the gel. Moreover, the microstructure material can be varied in terms of composition, stiffness and density and the microstructures may be made of either natural or synthetic polymers and can be non-degradable or biodegradable.

Due to their long history of use in clinical applications, poly(hydroxyortho esters) such as polyglycolic acid (PGA), poly lactic acid (PLA) and copolymers thereof are often used to fabricate three-dimensional porous scaffolds to support cell attachment, proliferation, migration, and extracellular matrix synthesis. The polymer may be a biodegradable polymer or a non-biodegradable polymer, but preferably is a biodegradable polymer. Biodegradable polymers include, but are not limited to collagen, elastin, hyaluronic acid and derivatives, sodium alginate and derivatives, chitosan and derivatives gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids). The biodegradeable polymers used herein may be copolymers of the above polymers as well as blends and combinations of the above polymers. (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986).

In particular preferred embodiments, the biodegradable or resorbable polymer is one that is formed from one or more monomers selected from the group consisting of lactide, glycolide, ε-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid), poly(L-lactide)/poly(D,-L-Lactide) blends or copolymers of L-lactide and D,L-lactide. In yet another aspect, the polymer includes a copolymer of lactide and e-caprolactone. In yet another aspect, the polymer includes a polyester (e.g., a poly(lactide-co-glycolide). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide; see e.g., U.S. Pat. No. 6,531,146 and U.S. application No. 2004/0137033.). Other examples of biodegradable materials include polyglactin, and polyglycolic acid.

Representative examples of non-biodegradable compositions include ethylene-co-vinyl acetate copolymers, acrylic-based and methacrylic-based polymers (e.g., poly(acrylic acid), poly(methylacrylic acid), poly(methylmethacrylate), poly(hydroxyethyl methacrylate), poly(alkylcynoacrylate), poly(alkyl acrylates), poly(alkyl methacrylates)), polyolefins such as poly(ethylene) or poly(propylene), polyamides (e.g., nylon 6,6), poly(urethanes) (e.g., poly(ester urethanes), poly(ether urethanes), poly(carbonate urethanes), poly(ester-urea)), polyesters (e.g., PET, polybutyleneterephthalate, and polyhexyleneterephthalate), olyethers (poly(ethylene oxide), poly(propylene oxide), poly(ethylene oxide)-poly(propylene oxide) copolymers, diblock and triblock copolymers, poly(tetramethylene glycol)), silicone containing polymers and vinyl-based polymers (polyvinylpyrrolidone, poly(vinyl alcohol), poly(vinyl acetate phthalate), poly(styrene-co-isobutylene-co-styrene), fluorine containing polymers (fluoropolymers) such as fluorinated ethylene propylene (FEP) or polytetrafluoroethylene (e.g., expanded PTFE).

The polymers may be combinations of biodegradable polymers, and also may be combinations of biodegradable and non-biodegradable polymers. Further examples of polymers that may be used are either anionic (e.g., alginate, carrageenin, hyaluronic acid, dextran sulfate, chondroitin sulfate, carboxymethyl dextran, caboxymethyl cellulose and poly(acrylic acid)), or cationic (e.g., chitosan, poly-I-lysine, polyethylenimine, and poly(allyl amine)) (see generally, Dunn et al., J. Applied Polymer Sci. 50:353, 1993; Cascone et al., J. Materials Sci.: Materials in Medicine 5:770, 1994; Shiraishi et al., Biol. Pharm. Bull. 16:1164, 1993; Thacharodi and Rao, Int'l J. Pharm. 120:115, 1995; Miyazaki et al., Int'l J. Pharm. 118:257, 1995). Preferred polymers (including copolymers and blends of these polymers) include poly(ethylene-co-vinyl acetate), poly(carbonate urethanes), poly(hydroxyl acids) (e.g., poly(D,L-lactic acid) oligomers and polymers, poly(L-lactic acid) oligomers and polymers, poly(D-lactic acid) oligomers and polymers, poly(glycolic acid), copolymers of lactic acid and glycolic acid, copolymers of lactide and glycolide, poly(caprolactone), copolymers of lactide or glycolide and ε-caprolactone), poly(valerolactone), poly(anhydrides), copolymers prepared from caprolactone and/or lactide and/or glycolide and/or polyethylene glycol.

### GELS FOR USE IN THE COMPOSITES OF THE INVENTION

A variety of polymeric gels that have been suggested for use in tissue engineering applications due to their ability to manipulate the material properties and ease of processing. In exemplary embodiments of the present invention, Matrigel (rich in ECM proteins) was used. As an alternative to Matrigel, hydrogels made from copolymers of poly(ethylene glycol) and poly(N-isopropylacrylamide) may be used. These polymers self-assemble to form gels of various architectures in a thermoreversible fashion. The polymers are block or star copolymers with a central hydrophilic poly(ethylene glycol) (PEG) segment and temperature responsive poly(N-isopropylacrylamide) (PNIPAAm) terminal segment. PEGS are known to be hydrophilic, biocompatible, and intrinsically resistant to protein adsorption and cell adhesion, and provide a “blank scaffold”, devoid of biological interactions. The values of elastic modulus, loss tangent, and yield strength can be changed by varying the ratios of the two monomers. At low temperature, they form liquid aqueous solutions with low to moderate injection viscosities, but form relatively strong elastic gels upon warming to physiological temperature, suitable for applications in cardiac tissue engineering. Other gels include collagen, fibrin, Hyaluronan, chitosan or other natural protein based gels or combinations thereof. Also, synthetic gels such as the hydrogels may be used.

In exemplary embodiments, the gel seeding protocol is based on methods described in Norman and Desai, 2005. Initially we use SU-8 (same material used for the freefloating microstructures because it is simpler to prepare and does not need plasma oxidation). Later we will use PDMS and biodegradable polymers. The PDMS polymer scaffold is slightly hydrophobic, impeding the gel solution from molding to the 3D features of the scaffold. Therefore, we improve the hydrophilic character of a surface by treatment with oxygen plasma, (Murakami, 1998). The scaffolds are then sterilized in an autoclave and plasma oxidized, if necessary, at 50 W for 15 min. They are then immediately placed in a sterile chamber until ready to add the gel matrix and cells to the scaffold. The spaces are filled with a primary ventricle cell mix of myocytes, fibroblasts and other cells in suspension in Matrigel with the concentrations of cells. The gel suspension containing the cells is pipetted over the microrod scaffold and molded around it by application of downward pressure. This technique is based on the pressure-assisted replication process (Folch et al., 2000). The thickness of the layer is the same or greater than microrod height.

### USES OF THE COMPOSITIONS OF THE INVENTION

As noted herein throughout the composites of the invention can be used as a three-dimensional cell culture system for the growth and proliferation of cells. Such cultures may be used for the study of the cells, for the production of further cells or for the production of protein products of the cells.

In other specific embodiments, the composites of the invention, i.e., the microstructures suspended in a gel may be seeded with the cells and molded or otherwise formed into flexible compositions that can be used as “patches.” Such “patches” may be comprised of just the cells of the patch so that they can act as cellular tissue patches or tissue grafts, or they may be impregnated or otherwise loaded with a drug or other biologically active agent to be delivered (e.g., in a controlled-release manner).

In specific embodiments, the composites three-dimensional culture systems of the present invention will be useful both as substrata for the growth and propagation of tissue cells that may be seeded on the substrata and also as implantable devices. In those embodiments where the cell culture system is used as an bioimplantable device, the substrate may be formulated into a shape suitable for implantation. For example, as described in U.S. Pat. No. 6,423,092 (incorporated herein by reference), it may be desirable to fabricate a biodegradable stent for implantation into a lumen. Organs for which tissue implantation patches may be generated include, but are not limited to skin tissue for skin grafts, myocardial tissue, bone tissue for bone regeneration, testicular tissue, endothelial cells, blood vessels, and any other cells from which a tissue patch may be generated. Thus, those of skill in the art would understand that the aforementioned organs/cells are merely exemplary organs/cell types and it should be understood that cells from any organ may be seeded onto the three dimensional matrices of the invention to produce useful tissue for implantation and/or study.

The patches made in the present invention may be combined with drugs for delivery or therapeutic agents that can form part of a tissue patch prepared from the polymers of the invention. For example, the three dimensional culture matrix may conform to tissue and release the agent (e.g., a therapeutic agent such as a drug or a diagnostic agent such as a marker, dye or other marker of that will allow visualization of a diseased state). In preferred examples, the microrods or other microstructures are prepared so as to have incorporated therein a drug, peptide, protein, or nucleic acid that can be released at a target site in a controlled release manner. See, e.g., U.S. Pat. No. 6,461,640. In certain embodiments, the microrods or microstructures may have incorporated therein an agent for delivery to the stressed heart may in order to prevent myocyte cell death and improve cardiac function. An exemplary such agent may be IGF-1 or mechano-growth factor, MGF (McCoy et al., J Physiol. Apr. 15, 1999;516 (Pt 2):583-92). In certain embodiments, it has been shown that it is possible to achieve an encapsulation efficiency of greater than 50% for hydrophobic peptides such as MGF using e.g., acetone or acetonitrile as the solvent. Acetonitrile was chosen as an ideal solvent for MGF and it is possible to achieve encapsulaton efficiencies of 60 to 80% in this manner. This means that for an initial drug concentration of 1 mg/ml, MGF loading of ˜500 μg/ml particle may be achieved. Given these measures of MGF loading in vivo and in vitro, and since the microrods can be loaded with 500 μg/ml, a solution of 2-10 μL would be sufficient to deliver effective dosages of the MGF. These preliminary calculations show that the microrods can be used to regenerate and strengthen the damaged myocardium in vivo.

The microrods or other microstructures may be impregnated with an antioxidant and/or antimicrobial. See, e.g., U.S. Pat. No. 6,572,878. The cell culture composite of the invention may be formulated into a tissue patch that is adapted to cover a bony dissection in the spine. See, e.g., U.S. Pat. No. 5,868,745 and U.S. patent application No. 2003/0078588. The tissue patch made of the compositions of the invention may be prepared to be wrapped around a nerve in a canal to reduce fibroplasia. See, e.g., U.S. Pat. No. 6,106,558. The tissue patch maybe a resorbable collagen membrane that is wrapped around the spinal chord to inhibit cell adhesions. See, e.g., U.S. Pat. No. 6,221,109. The tissue patch may be used as a dressing to cover a wound and promote wound healing. See, e.g., U.S. Pat. No. 6,548,728.

The patches prepared herein may be made sterile either by preparing them under aseptic environment and/or they may be terminally sterilized using methods known in the art, such as gamma radiation or electron beam sterilization methods or a combination of both of these methods. Such patches may be applied to any bodily conduit or any tissue.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus are considered to constitute certain aspects for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The following example describes techniques used in the present invention.

Microrod Fabrication. The microrods were created in SU-8 negative photoresist. Microrods were designed to be approximately 100 μm long with a 15 μm×15 μm cross-section. A 3 inch silicon wafer was first cleaned in piranha solution (3:1 H2SO4:H2O2) for 20 min then rinsed with deionized water and baked at 200° C. for 2 min on a hot plate to dehydrate the surface. The wafers were rinsed with acetone, methanol, and isopropanol immediately prior to coating. SU-8 2010 (Microchem Inc., Newton, Mass.) was spun onto the wafer surface to a thickness of 15 μm. After the prebake (2 min at 65° C., 4 min at 95° C.), the wafer was exposed using a Karl Suss MJB3 mask aligner to a 365 nm light source through a transparency mask (5 mW/cm2 for 1 min). The wafer was then post-baked (1 min at 65° C., 1 min at 95° C.), and developed in SU-8 developer (Microchem Inc., Newton, Mass.) until uncrosslinked photoresist was removed. The height of the microrods was determined using a Tencor Alpha Step profilometer. Length and width measurements were made from light microscope images.

Microrods were removed from the wafer using a razor blade and soaked in 70% ethanol for 1 hr to sterilize. Microrods were pelleted in a centrifuge (5000 RPM for 10 min) then rinsed with sterile 1×Phosphate Buffered Saline (PBS). This rinsing process repeated three times). The microrods were finally resuspended in cold, serum free cell culture media containing 1% penicillin/streptomycin solution.

2.2. SU-8 2D-film fabrication. 2D SU-8 films were used to determine if the surface chemistry had any impact on fibroblast proliferation rate. The processing of the wafer was exactly the same as with the fabrication of the microrods, however, no photomask was used and the entire surface was exposed to UV-light. This created an unpatterned 15 μm thick SU-8 film on the silicon wafer. The wafer was then diced into 1 cm² pieces.

Microrod-Matrigel composite matrix. Matrigel was used for suspending cells and microrods. Matrigel simulates a natural 3D ECM environment. Matrigel is a commercially available, solubilized basement membrane extracted from EHS mouse sarcoma that is rich in ECM proteins and provides a natural ECM environment that supports cell growth [Kleinman et al., Biotechnol. Bioeng. 200693(6):1060-1068]. Dilute Matrigel (BD Biosciences, San Jose Calif.) was made in the following manner. A sterile vial for mixing of reagents was placed on ice. Matrigel stock solution that had been stored at 4° C. was placed in the vial. To this was added enough microrod solution, cell suspension and cold serum free media so that the final concentration of Matrigel was either 4.0 mg/mL or 3.37 mg/mL and contained 78,000 cells/mL and either 7,800 or 780 microrods/mL (a 10:1 and 100:1 ratio of cells-to-microrods). Controls were created by replacing the microrod solution with serum free media. This solution was then mixed gently by pipetting it up and down several times. The vials were placed into an incubator at 37° C. for 10 min to partially set the gel. The gels were mixed once more to prevent settling of microrods. 3D gels were made by placing 64 μL of this solution into individual wells of a 96-well plate resulting in a 2 mm thick gel. Plates were placed in an incubator at 37° C. for 30 min to allow the gels to set. Each gel was then covered with 5.4 mm of media and incubated at 37° C. (5% CO2 atmosphere). Media was changed daily. Each 64 μL gel contained approximately 5,000 cells and either 50 or 500 microrods.

Rheology of gels. The addition of microrods to the Matrigel results in a short-fiber composite matrix. The dynamic shear modulus, G′, and loss modulus, G″, of gels containing microrods at 0, 50 and 500 rods per 64 μL gel (volume fraction of the microrods in gels: 0, 0.00003 and 0.0003, respectively) were assessed between 0.1 and 5 Hz using a parallel plate rheometer with a gap of 1 mm between plates and a force of 0.1 N (n=10). Testing was done on both concentrations of Matrigel.

Primary Fibroblast Cell Culture: Primary Neonatal Rat Ventricular Fibroblasts (NRVF) were isolated as described previously [Boateng et al., Am J Physiol Cell Physiol July 2003;285(1):C171-182.]. Primary NRVF were cultured in a 75 cm2 tissue culture flask using a growth medium of Dulbecco's Minimum Essential Medium (DMEM) supplemented with 10% Fetal Bovine Serum (FBS) and antimycotic/antibiotic solution. Media was changed every 2-3 days until confluence of NRVF, approximately 1 week after initial plating. Cells were passaged once by detaching from the flask in 0.25% trypsin in DMEM with EDTA and replated until they were near confluence again (2-3 days). NRVF were detached, centrifuged at 3000 RPM for 6 min and then resuspended in serum free, cold, DMEM containing the antimycotic/antibiotic solution.

Fibroblast Cell Line Cell Culture. For this study IMR-90 human lung fibroblasts (ATCC, Manassas, Va.) were used. IMR-90 cells are a myofibroblast (fibroblasts with smooth muscle like properties [Ehler et al., Cell Motil Cytoskeleton 1996;34(4):288-298]) cell line that is commercially available, and so are a convenient source of fibroblasts for culture. Cells were grown in a 75 cm² tissue culture flask using a growth medium of Eagle's Minimum Essential Medium (EMEM) (ATCC, Manassas, Va.) supplemented with 10% FBS and 1% penicillin/streptomycin solution. Media was changed every 3-4 days. When ready for use, the cells were detached from the flask with 0.25% trypsin/EDTA, diluted in serum free EMEM at 3:1 of the trypsin volume, centrifuged at 2000 RPM for 2 min and then resuspended in cold, serum free EMEM containing 1% penicillin/streptomycin solution. Cells were used between population doubling levels of 27-33.

Proliferation of fibroblasts on SU-8 2D films. SU-8 films coated on 1 cm² pieces of silicon wafer were placed in 24-well plates. Cells were added to the wells at a concentration of 18,500 cells/cm² (n=3). Proliferation was assessed at 1, 2 and 5 days after seeding using WST-1 (Roche Applied Sciences, Indianapolis, Ind.). The stable tetrazolium salt WST-1 is cleaved to a soluble formazan by a complex cellular mechanism that occurs primarily at the cell surface. This bioreduction is largely dependent on the glycolytic production of NAD(P)H by mitochondrialsuccinate-tetrazolium-reductase system in viable cells. Therefore, the amount of formazan dye formed directly correlates to the number of metabolically active cells. The reduced formalin dye is water soluble and can be read spectrophotometrically. Films were removed from the culture plate wells, placed in clean wells and covered with 1 mL of media and 10 μL of WST-1. The plates were incubated at 37° C. for 2 hrs. After 2 hrs, the films were removed from the wells and the net absorbance of the remaining solution was taken as 450 nm-630 nm. The proliferation results were compared to that of fibroblasts growing in control 3D Matrigel gels. Absorbance values were normalized to the number of cells present after 24 hr of culture.

Proliferation of IMR-90 and NRVF cells in 3D gels. Cellular proliferation was assessed at 1, 2 and 5 days after initial seeding in the gels using the WST-1 assay. An absorption standard curve first was determined for fibroblasts growing in 3D Matrigel gels of the same concentration and thickness as used in the proliferation experiment. This allowed for the determination of the linear range of absorption for experimental cell concentrations. Final readings were fit to this curve to determine final cell counts in the gels. Also, the effect on the absorbance, due to the inclusion of microrods, was assessed. The inclusion of microrods at the concentrations used does not significantly increase the absorbance at either wavelength. Any changes in absorbance during the proliferation assay, therefore, can be attributed strictly to changes in the number of cells present in the gel.

Cells were initially seeded at a density of 5,000 cells per 64 μL gel with both 100:1 and 10:1 cell:microrod ratios. Control gels contained no microrods. On the day of the assay, the media was removed from the wells and was replaced with 100 μL of fresh growth media and 16.4 μL of the WST-1 reagent. The WST-1 volume represents 10% of the total volume: 100 μL of media and 64 μL of the gel scaffold. The plates were wrapped in foil and placed on a shaker plate for 1 min. They were then placed back in the incubator for 4 hrs. After this time, the absorbance of the wells was read at 450 nm with a baseline of 630 nm using a plate reader. The net absorbance (450 nm-630 nm) was compared to a standard curve to determine the number of cell present (n=4). For neonatal rat ventricular fibroblasts, NRVF, the mean absorbance (450 nm-630 nm for the baseline) of each experimental group was normalized to the mean absorbance of Matrigel alone on each day (n=4).

Fluorescent staining of the F-actin cytoskeleton and fibroblast nuclei. The F-actin cytoskeleton was fluorescently stained with rhodamine-phalloidin. After rinsing with PBS, gels were soaked in 1% Triton-X 100 (Sigma-Aldrich) for 2 min to permeabilize the cells. The Triton-X solution was removed and the gels were rinsed again using PBS. Gels were covered with a 0.33 ∞M rhodamine-phalloidin (Invitrogen, Carlsbad, Calif.) solution for 30 min. Gels were rinsed with PBS before imaging. Images were taken by focusing on a plane in the gel with a fluorescent microscope.

Nuclei of IMR-90 cells in composite gels were stained with 10 μM Hoechst 33342 (Invitrogen, Carlsbad, Calif.). Media was removed from the wells and gels were rinsed with PBS. The gels were then covered with Hoechst dye and placed in the incubator for 30 minutes. After this time the dye was removed from gels and they were rinsed again with PBS.

Microscopy. Confocal microscopy was performed to visualize the distribution and organization of microrods in three-dimensions. Microscopy was done using an Olympus Fluoview 1000 confocal scanning microscope equipped with Fluoview software. The sample was excited with a 405 nm laser. SU-8 fluoresces blue when excited at 405 nm making the microrods easy to visualize. Confocal microscopy images were processed using ImageJ software [Grish et al., Indian J Cancer January-March 2004;41(1):47].

Scanning electron microscopy (SEM) of microrods on a piece of silicon wafer was performed using a JEOL JSM 6100 Scanning Electron Microscope with an acceleration velocity of 5 kV. Samples were sputter-coated with gold to a thickness of 20 nm (Cressington 108 sputter coater).

Fluorescent images were taken using an Olympus BX60 upright microscope. Microrod and IMR-90 images were analyzed using Axiovision software (Release 4.3).

### Example 2

### Results and Discussion

The microrods were ellipsoidal in shape and measured 15.2±0.3 μm high, with a top surface area of 2463.8±42.5 μm2. The lengths of the major and minor axes were 120.9±0.7 μm and 26.2±0.2 μm, respectively. After mixing the microrods into the Matrigel and allowing it to set, the microrods appear to have a random organization and distribution throughout the gel.

A few techniques have recently been demonstrated for spatially patterning the internal structure of a gel. Both single-photon and two-photon absorption photolithography allow for changing the local cross-link density and hence the local stiffness and porosity in photosensitive poly(ethylene glycol) (PEG) gels [Hahn et al., Advanced Materials, 2006;18(20):2679-2684]. Electrophoretic placement with subsequent photo-entrapment in PEG gels [Albrecht et al., Lab Chip January 2005;5(1):111-118], and laser guided placement of cells [Nahmias et al., Biotechnol Bioeng Oct. 20, 2005;92(2):129-136] have also been demonstrated for patterning cells with a 3D matrix without the use of internal features. However, Nahmias et al. [Biotechnol Bioeng Oct. 20, 2005;92(2):129-136] have shown that the cells fail to maintain long-term pattern integrity without a guiding extracellular force. Light irradiating techniques are problematic because they require a photoactive gel with good optical clarity and seeding with cells prior to crosslinking can cause cells to be damaged by the wavelengths and energy levels used [Black et al., J Photochem Photobiol B August 1997;40(1):29-47]. Further viability issues revolve around the presence of free-radicals and use of a photoinitiator necessary for crosslinking PEG [Bryant et al., J Biomater Sci Polym. Ed 2000;11(5):439-457].

The approach applied in invention allows for the use of a biological matrix providing a more natural environment for the cells. Pre-seeding the gel with cells in the presence of the microfabricated features is not a problem because harsh chemicals or irradiation are not needed to set the gel. Also, photolithographic patterning of embeddable features allows for high-throughput, parallel fabrication of microstructures. The mask used for the microrods produced approximately 115,000 microrods on one 3 inch silicon wafer. Also, SU-8 can be further surface modified through reaction with free epoxy groups on its surface allowing for biochemical as well as mechanical cues to be presented making this method a powerful technique to alter not only the mechanical but also the biochemical microenvironment [Wang et al., Langmuir Mar. 14, 2006;22(6):2719-2725].

Rheology of gels containing microrods. The addition of microrods to the 3D gel results in a short-fiber composite. Addition of short-fibers can dramatically change the stiffness of the matrix phase. It has been shown that changes in the stiffness of biological matrices can significantly change cell proliferation [Ghosh et al., Biomaterials February 2007;28(4):671-679]. To ensure that the bulk properties of the gels were not changed by the inclusion of microrods, a parallel plate rheometer was used to examine gels with varying concentrations of microrods. The values of G′ and G″ at the small volume fractions of microrods used were not significantly different from Matrigel with no microrods. The moduli remain fairly constant up to 5 Hz. The values of G′ for 4.0 mg/mL and the 3.37 mg/mL gels, at 1 Hz, were 22.9 PA and 1.3 Pa, respectively. The higher concentration of Matrigel provides a stiffer gel for the cells. The value of G′ for the 4.0 mg/mL gel is similar to that reported previously [Semler et al., Biotechnol Bioeng Aug. 20, 2000;69(4):359-369]. In contrast the dynamic shear modulus of SU-8 films has been reported at 1.66 GPa [Hossenlopp et al., Journal of Polymer Science Part B: Polymer Physics 2004;42(12):2373-2384]. The microrods, therefore, provide a discrete zone within the gel that is 6 orders of magnitude stiffer than the surrounding gel.

Proliferation of IMR-90 fibroblasts on SU-8 films. In order to assess the affects of surface chemistry of the SU-8 on fibroblast proliferation, the proliferation rate of IMR-90 fibroblasts on flat SU-8 films was compared to that of cells in Matrigel over a 5 day period. Fibroblasts growing on SU-8 films proliferated at the same rate as those growing in 3D Matrigel. Both materials showed an initial lag in start of proliferation at 2 days culture. By day 5 the fractional increase in cell number was the same for both conditions. Thus, differences in material surface chemistry on observed changes in cell growth could be ruled out.

Proliferation of fibroblasts in 3D gels. The fractional increase in absorbance of WST-1 above baseline for Matrigel alone was used to assess NRVF proliferation as a function of Matrigel concentration and cell:microrod ratio per well. NRVF proliferation is significantly (P<0.05) reduced in lower concentration gels (3.37 mg/mL, 1.3 Pa) containing a ratio of cells-to-microrods of both 100:1 and 10:1. In the stiffer Matrigel (4.0 mg/ml, 22.9 Pa) without microrods, NRVF proliferation was insignificant over the period investigated.

Fluorescent staining of the F-actin cytoskeleton. Staining of the F-actin cytoskeleton with rhodamine-phalloidin shows actin bundles terminating where cells attach to microrods. It could readily be seen that a cell bridges several microrods. Formation of stress fibers are observed between points of contact to the microrods.

Nuclear staining with Hoechst 33342 shows that attachment to the microrod causes the nucleus of the cell to distort in shape. For cells attached to microrods, nuclear positioning was shifted towards the microstructure and nuclear shape was distorted to conform to the microrod surface. Nuclei of attached cells also appear larger than those of unattached cells. These observations are similar to what has been observed in micropatterned 2D structures.

The behavior of cells in tissues is strongly modulated by mechanical forces. Mechanotransduction of extracellular forces encountered by cells in their environment are important in tissue morphogenesis and have been shown to affect a number of different cellular processes: assembly and organization of the extracellular matrix, gene transcription, cell motility, growth, differentiation, apoptosis, and signal transduction. Change of nuclear shape via displacement of organelles caused by external application of forces from the ECM to the interior of the cell through the cytoskeleton has also been proposed as a mechanism by which fibroblasts can alter their proliferation.

From the above studies, it has been shown that microfabricated structures can be used to influence the growth of cells in a 3D matrix. Specifically, the addition of microrods into a 3D biological matrix retards fibroblast proliferation. The ability of SU-8 surface chemistry alone to stunt fibroblast proliferation was niled out as was any change in bulk rheological properties of the gel. The modulation of proliferative activity appears to be directly related to the presence of microstructures in the gel and the interaction of cells with these microstructures.

Gaining the ability to control the forces individual cell sense in a 3D matrix will be a powerful tool in controlling tissue growth in vitro. However, feature generation and positioning in 3D is challenging at the microscale. This microrod composite system allows for local control over microenvironmental cues in an extracellular matrix. It is a simple and versatile fabrication method that allows for incorporation into any synthetic or natural matrix. Moreover, such a microrod system can be readily injected in vivo allowing for future therapeutic potential.

We claim:

1. A three-dimensional cell culture matrix comprising composite of a cell culture gel and polymeric microstructures suspended in said cell culture gel, wherein said microstructures have a cross sectional area of between 1 μm² to 2500 μm² and are randomly suspended in said cell in a concentration of from 10 to 100,000 microstructures/ml of gel, wherein the microstructures form a scaffold for the cells.

2. The three-dimensional cell culture matrix of claim 1, wherein said cell culture gel is selected from the group consisting of Matrigel, an alginate gel, a collagen gel, and an agarose gel, or a synthetic hydrogel.

3. The three-dimensional cell culture matrix of claim 2, wherein the gel further comprises one or more of laminin, fibrin, fibronectin, proteoglycans, glycoproteins, glycosaminoglycans, chemotactic agents, or growth factors.

4. The three-dimensional cell culture matrix of claim 1, wherein the microstructures are comprised of non-biodegradeable or biodegradeable polymers.

5. The three-dimensional cell culture matrix of claim 1, wherein the microstructures are comprised of collagen-GAG, collagen, fibrin, PLA, PGA, PLA-PGA co-polymers, poly(anhydrides), poly(hydroxy acids), poly(ortho esters), poly(propylfumerates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), co-polymers of the above, mixtures of the above, and adducts of the above.

6. The three dimensional cell culture matrix of claim 4, wherein substantially all of the microstructures are comprised of a single polymer.

7. The three dimensional cell culture matrix of claim 4 wherein the microstructures form a mixed population of microstructures made of different polymers.

8. The three-dimensional cell culture matrix of claim 1 wherein substantially all the microstructures are of a uniform size and shape.

9. The three-dimensional cell culture matrix of claim 1 wherein the microstructures are of a different sizes and shapes.

10. The three-dimensional cell culture matrix of claim 1 wherein the microstructures comprise a mixture of poly(L-lactic acid) and poly(lactic acid-co-glycolic acid).

11. The three-dimensional cell culture matrix of claim 1 wherein the microstructures comprise materials of different stiffnesses.

12. The three-dimensional cell culture matrix of claim 1 wherein one or more of the microstructures further comprise a cell-adhesion promoting agent on the surface of the microstructures.

13. The three-dimensional cell culture matrix of claim 11, wherein said agent is selected from the group consisting of fibronectin, integrins, extracellular matrix or peptides, and oligonucleotides that promote cell adhesion.

14. The three dimensional cell culture matrix of claim 1, wherein said gel further comprises agents to facilitate cell adhesion and cell growth selected from the group consisting of further comprises one or more of laminin, fibrin, fibronectin, proteoglycans, glycoproteins, glycosaminoglycans, chemotactic agents, or growth factors.

15. The three-dimensional cell culture matrix of claim 14, wherein the growth factor is selected from activin-A (ACT), retinoic acid (RA), epidermal growth factor, bone morphogenetic protein, TGF-β, hepatocyte growth factor, platelet-derived growth factor, TGF-β, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), mechano-growth factor (MGF), muscle morphogenic factor and stem cell growth factors.

16. The three-dimensional cell culture matrix of claim 1 wherein said microstructure has encapsulated therein an agent to be delivered to a target site in vivo.

17. The three-dimensional cell culture matrix of claim 1 wherein the gel comprises 50,000 microstructures per ml.

18. The three-dimensional cell culture matrix of claim 1 wherein the microstructures are microrods having dimensions of 100 μm×15 μm×15 μm.

19. The three-dimensional cell culture matrix of claim 1 wherein the ratio of microstructures to cells is between one to one and 100 to one.

20. A cell culture of system comprising a population of cells to be cultured seeded in the gel of a three-dimensional matrix according to any of claims 1-19.

21. The cell culture system of claim 20, wherein said cells are selected from the group consisting of endothelial cells, myoblasts, cardiomyocytes, stem cells, skeletal muscle cells, smooth muscle cells, fibroblasts, a human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cel, a xenogenic cell, an allogenic cell, an adult stem cell, and a post-natal stem cell.

22. The cell culture system of claim 20, wherein the cells are seeded at an initial concentration of from about 250×103 cells/μm³ gel to about 1000×10³ cells/μm³ gel.

23. A method of growing cells in culture comprising: seeding a population of cells in the gel of a three-dimensional matrix according to any of claims 1-20 and culturing the seeded cells in a cell culture medium that supports the growth of the cells of said population.
